(19) United States
(12) Patent Application Publication
(10) Pub. No.: US 2025/0090182 A1
(43) Pub. Date: Mar. 20, 2025

(54) MULTI-LUMEN ASPIRATION CATHETERS, AND ASSOCIATED SYSTEMS AND METHODS

(71) Applicant: Inari Medical, Inc., Irvine, CA (US)

(72) Inventors: Kali Wen-Tseng Slaughter, Santa Ana, CA (US); Kendall Anne Ruggles, New Port Beach, CA (US); Benjamin Edward Merritt, San Clemente, CA (US); Miguel Rendon Flores, Fallbrook, CA (US)

(21) Appl. No.: 18/885,201

(22) Filed: Sep. 13, 2024

Related U.S. Application Data
(60) Provisional application No. 63/582,610, filed on Sep. 14, 2023.

(51) Int. Cl. A61B 17/22; A61B 17/00

(52) U.S. Cl. CPC A61B 17/22; A61B 17/00234; A61B 2017/00238; A61B 2017/00305; A61B 2017/00544; A61B 2017/00561; A61B 2017/22079; A61B 2217/005; A61B 2217/007

(57) ABSTRACT

Disclosed herein are clot treatment systems including catheters having multiple lumens, and associated systems and methods. In some embodiments, a clot treatment system includes a catheter having a distal tip configured to be intravascularly positioned proximate to clot material within a blood vessel. The catheter can further include/define an aspiration lumen having a distal aspiration opening and an injection lumen having a distal injection opening. The clot treatment system can further include a pressure source fluidly coupled to the aspiration lumen, and a fluid source fluidly coupled to the injection lumen. The pressure source is configured to generate negative pressure within the aspiration lumen to aspirate at least a portion of the clot material through the distal aspiration opening. The fluid source is configured to inject a fluid through the injection lumen and out of the distal injection opening into the blood vessel.

### CROSS-REFERENCE TO RELATED APPLICATION

This application claims the benefit of U.S. Provisional Patent Application No. 63/582,610, filed Sep. 14, 2023, and titled “MULTI-LUMEN ASPIRATION CATHETERS, AND ASSOCIATED SYSTEMS AND METHODS,” which is incorporated herein by reference in its entirety.

### TECHNICAL FIELD

The present technology generally relates to clot treatment systems including catheters having a primary aspiration lumen for aspirating clot material from within a blood vessel and a separate secondary injection lumen for injecting fluid into the blood vessel.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. Infarction does not develop or is greatly limited if the flow of blood is reestablished rapidly. Failure to reestablish blood flow can lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT arises most commonly when there is a propensity for stagnated blood (e.g., long distance air travel, immobility, etc.) and clotting (e.g., cancer, recent surgery, such as orthopedic surgery, etc.). DVT causes harm by: (1) obstructing drainage of venous blood from the legs leading to swelling, ulcers, pain, and infection, and (2) serving as a reservoir for blood clots to travel to other parts of the body including the heart, lungs, brain (stroke), abdominal organs, and/or extremities.

In the pulmonary circulation, the undesirable material can cause harm by obstructing pulmonary arteries-a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

There are many existing techniques to reestablish blood flow through an occluded vessel. One common surgical technique, an embolectomy, involves incising a blood vessel and introducing a balloon-tipped device (such as the Fogarty catheter) to the location of the occlusion. The balloon is then inflated at a point beyond the clot and used to translate the obstructing material back to the point of incision. The obstructing material is then removed by the surgeon. Although such surgical techniques have been useful, exposing a patient to surgery may be traumatic and best avoided when possible. Additionally, the use of a Fogarty catheter may be problematic due to the possible risk of damaging the interior lining of the vessel as the catheter is being withdrawn.

Percutaneous methods are also utilized for reestablishing blood flow. A common percutaneous technique is referred to as balloon angioplasty where a balloon-tipped catheter is introduced to a blood vessel (e.g., typically through an introducing catheter). The balloon-tipped catheter is then advanced to the point of the occlusion and inflated to dilate the stenosis. Balloon angioplasty is appropriate for treating vessel stenosis, but it is generally not effective for treating acute thromboembolisms as none of the occlusive material is removed and the vessel will re-stenos after dilation. Another percutaneous technique involves placing a catheter near the clot and infusing streptokinase, urokinase, or other thrombolytic agents to dissolve the clot. Unfortunately, thrombolysis typically takes hours to days to be successful. Additionally, thrombolytic agents can cause hemorrhage and in many patients the agents cannot be used at all.

Various devices exist for performing a thrombectomy or removing other foreign material. However, such devices have been found to have structures which are either highly complex, cause trauma to the treatment vessel, or lack sufficient retaining structure and thus cannot be appropriately fixed against the vessel to perform adequately. Furthermore, many of the devices have highly complex structures that lead to manufacturing and quality control difficulties as well as delivery issues when passing through tortuous or small diameter catheters. Less complex devices may allow the user to pull through the clot, particularly with inexperienced users, and such devices may not completely capture and/or collect all the clot material.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a clot treatment system in accordance with embodiments of the present technology.

FIG. 2A is a cross-sectional view of a catheter of the clot treatment system of FIG. 1 taken along the line 2A-2A in FIG. 1 in accordance with embodiments of the present technology.

FIGS. 2B-2D are proximally-facing perspective views of a distal portion of the catheter of FIGS. 1 and 2A with a dilator of the clot treatment system of FIG. 1 inserted therein in accordance with embodiments of the present technology.

FIG. 3 is a cross-sectional view of the catheter of FIG. 1 taken along the line 3-3 in FIG. 1 in accordance with additional embodiments of the present technology.

FIG. 4 is a proximally-facing perspective end-on view of the catheter of FIGS. 1 and/or 3 in accordance with additional embodiments of the present technology.

FIG. 5A is a cross-sectional view of the catheter of FIG. 1 taken along the line 5A-5A in FIG. 1 in accordance with additional embodiments of the present technology. FIG. 5B is a proximally-facing perspective end-on view of the catheter of FIGS. 1 and 5A in accordance with embodiments of the present technology.

FIG. 6 is a cross-sectional view of the catheter of FIG. 1 taken along the line 6-6 in FIG. 1 in accordance with embodiments of the present technology.

FIG. 7 is a flow diagram of a method or process of treating clot material within a patient in accordance with embodiments of the present technology.

FIGS. 8A-8C are sides views of a distal region of the catheter of the clot treatment system of FIG. 1 at various stages of the method of FIG. 7 in accordance with embodiments of the present technology.

FIG. 9 is a partially-schematic side view of the clot treatment system of FIG. 1 configured as a clot aspiration and filtered blood reinfusion system in accordance with embodiments of the present technology.

### DETAILED DESCRIPTION

The present technology is generally directed to clot treatment systems including catheters (e.g., aspiration catheters) having multiple lumens (e.g., dual lumens), and associated systems and methods. In some embodiments, a clot treatment system in accordance with the present technology includes a catheter having a distal tip configured to be intravascularly positioned proximate to clot material within a blood vessel. The catheter can further include/define an aspiration lumen having a distal aspiration opening and an injection lumen having a distal injection opening. The clot treatment system can further include a pressure source fluidly coupled to the aspiration lumen, and a fluid source fluidly coupled to the injection lumen. The pressure source is configured to generate negative pressure within the aspiration lumen to aspirate at least a portion of the clot material through the distal aspiration opening. The fluid source is configured to inject a fluid through the injection lumen and out of the distal injection opening into the blood vessel. In some embodiments, the distal injection opening is positioned proximal to the distal aspiration opening along the catheter.

The fluid can be a contrast fluid that permits visualization of a distal portion of the catheter including the distal tip under fluoroscopic imaging. The contrast fluid can enable an operator of the clot treatment system (e.g., a physician) to visualize a cause of occlusion of the aspiration lumen after aspiration. For example, the aspiration lumen may become occluded if (1) the distal tip of the catheter is positioned against a wall of the blood vessel and/or the blood vessel collapses during aspiration or (2) the clot material is not fully ingested into the distal tip and clogs the aspiration lumen. If the aspiration lumen is occluded by the wall of the blood vessel, the same or a different fluid can be injected through the injection lumen into the blood vessel to fill the blood vessel and/or the catheter can be retracted proximally until the aspiration lumen is no longer occluded (e.g., becomes unstuck from the wall of the vessel). If the aspiration lumen is occluded by the clot material, the catheter can be retracted proximally with the clot material captured thereby (e.g., stuck to the distal tip) from within the blood vessel and out of the patient.

Accordingly, the present technology permits the operator to directly visualize and determine the cause of an occlusion within the aspiration lumen (e.g., whether the catheter is engaged with and occluded by the wall of the blood vessel or occluded by clot material) via contrast fluid injected through the separate injection lumen. In contrast, many conventional clot removal techniques do not permit determination of the cause of an occlusion. For example, under fluoroscopy it can be difficult or impossible to tell what the cause of occlusion is because the area around the distal tip of the catheter (e.g., what the catheter is engaging) is not visible. In such systems, the catheter must be fully removed from the patient regardless of the cause of occlusion, cleaned, and subsequently reintroduced into the blood vessel. Using the present technology, if the catheter is merely engaged with the wall of the blood vessel, the operator can inject fluid through the injection lumen and/or slightly retract the catheter to release the wall of the blood vessel from the catheter and clear the occlusion. Notably, the operator need not retract the catheter fully from the patient to clear the occlusion-reducing procedure time. Additionally, the injection lumen is separate from the aspiration lumen such that fluid injection through the injection lumen minimizes the risk of reintroducing clot material into the blood vessel of the patient.

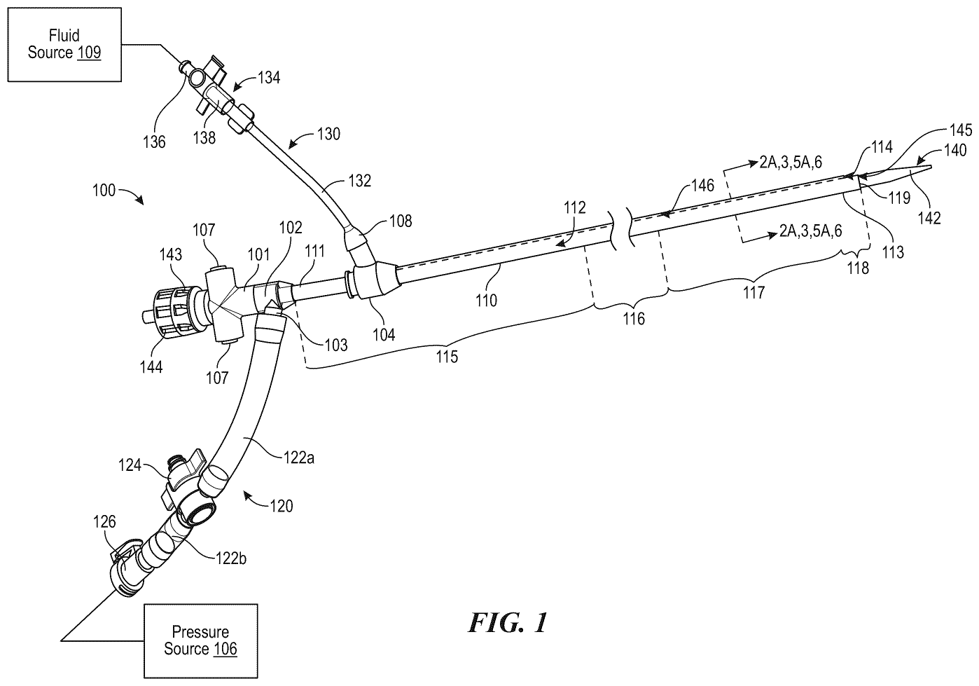

Certain details are set forth in the following description and in FIGS. 1-8C to provide a thorough understanding of various embodiments of the present technology. In other instances, well-known structures, materials, operations, and/or systems often associated with intravascular procedures, clot removal procedures, catheters, fluoroscopic or other medical imaging techniques, and the like are not shown or described in detail in the following disclosure to avoid unnecessarily obscuring the description of the various embodiments of the technology. Moreover, although reference is primarily made to aspiration catheters and catheters for use in clot removal procedures, the catheters of the present technology can be other types of catheters and/or can be used in other types of medical procedures. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

Moreover, although many of the devices and systems are described herein in the context of removing and/or treating clot material, the present technology can be used to remove and/or treat other unwanted material in addition or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, catheter 110 is first introduced and discussed with reference to FIG. 1A.

As used herein, unless expressly indicated otherwise, the terms “about,” “approximately,” “substantially” and the like mean within plus or minus 10% of the stated value. To the extent any materials incorporated herein by reference conflict with the present disclosure, the present disclosure controls. The headings provided herein are for convenience only and should not be construed as limiting the subject matter disclosed.

### I. SELECTED EMBODIMENTS OF CLOT TREATMENT SYSTEMS

FIG. 1 is a partially schematic side view of a clot treatment system 100 in accordance with embodiments of the present technology. The clot treatment system 100 can also be referred to as an aspiration assembly, an aspiration and fluid injection assembly, a clot removal system, a thrombectomy system, and/or the like. In the illustrated embodiment, the clot treatment system 100 includes a catheter 110 (e.g., an elongate member, a tube, a sheath, a shaft, and/or the like) having a proximal end portion 111 and a distal end portion 113. The catheter 110 further defines an aspiration lumen 112 (e.g., a first lumen, a primary lumen, a vacuum lumen, and/or the like) and one or more injection lumens 114 (e.g., a second lumen, a secondary lumen, a fluid injection lumen, an infusion lumen, and/or the like). The clot treatment system 100 further includes a first tubing assembly 120 fluidly coupled to the aspiration lumen 112 via a first port or first connector 102 and a second tubing assembly 130 fluidly coupled to the injection lumen 114 via a second port or second connector 104. The first connector 102 can be coupled to the proximal end portion 111 of the catheter 110 and a valve 101 can be fluidly coupled to a proximal portion of the first connector 102.

The valve 101 is fluidly coupled to the aspiration lumen 112 of the catheter 110 via the first connector 102. In some embodiments, the valve 101 is a hemostasis valve that is configured to maintain hemostasis during a clot removal procedure by inhibiting or even preventing fluid flow in the proximal direction through the valve 101 as various components such as delivery sheaths, pull members, guidewires, interventional devices, other aspiration catheters, and so on are inserted through the valve 101 to be delivered through the aspiration lumen 112 of the catheter 110 to a treatment site in a blood vessel. In some embodiments, the valve 101 can be a valve of the type disclosed in U.S. Pat. No. 11,000,682, filed Aug. 30, 2018, and titled “HEMOSTASIS VALVES AND METHODS OF USE,” which is incorporated herein by reference in its entirety. For example, the valve 101 can include one or more actuators 107 (e.g., buttons) that are biased to a position in which a lumen of the valve 101 is closed and that are actuatable (e.g., depressible) by a user to open the lumen of the valve 101. The first connector 102 includes a branch or side port 103 positioned to fluidly couple the aspiration lumen 112 of the catheter 110 to the first tubing assembly 120.

In the illustrated embodiment, the first tubing assembly 120 fluidly couples the aspiration lumen 112 of the catheter 110 to a pressure source 106 (which can also be referred to as a source of negative pressure, an aspiration source, and/or the like), such as a syringe, electric pump, and/or the like. The first tubing assembly 120 can include one or more tubing sections 122 (individually labeled as a first tubing section 122a and a second tubing section 122b), at least one fluid control device 124 (e.g., a valve), and at least one third connector 126 (e.g., a Toomey tip connector) for fluidly coupling the tubing assembly 120 to the pressure source 106 and/or other suitable components. In some embodiments, the fluid control device 124 is a stopcock that is fluidly coupled to (i) the side port 103 of the first connector 102 via the first tubing section 122a and (ii) the third connector 126 via the second tubing section 122b. The fluid control device 124 is externally operable by a user to regulate the flow of fluid therethrough and, specifically, from the aspiration lumen 112 of the catheter 110 to the pressure source 106. For example, the fluid control device 124 can be moved to a closed position to fluidly disconnect the pressure source 106 from the aspiration lumen 112 and moved to an open position to fluidly connect the pressure source 106 to the aspiration lumen 112. In some embodiments, the third connector 126 is a quick-release connector (e.g., a quick disconnect fitting) that enables rapid coupling/decoupling of the catheter 110 and the fluid control device 124 to/from the pressure source 106.

In the illustrated embodiment, the second connector 104 is coupled to the catheter 110 distal of the proximal end portion 111. The second connector 104 can include a branch or side port 108 positioned to fluidly couple the injection lumen 114 of the catheter 110 to the second tubing assembly 130. In the illustrated embodiment, the second tubing assembly 130 fluidly couples the injection lumen 114 of the catheter 110 to a fluid source 109. The fluid source 109 can include/contain a fluid such as contrast (e.g., a dye loaded with particles that are visible under fluoroscopy), saline, blood and/or another fluid and, in some embodiments, includes a pressure for forcing the fluid through the injection lumen 114 (e.g., for injecting the fluid through the injection lumen 114). For example, the fluid source 109 can comprise a syringe containing a contrast fluid, saline, heparin, anti-coagulant medications, and/or the like, and/or various mixtures thereof. The second tubing assembly 130 can include one or more tubing sections 132 coupled to a connector assembly 134. The connector assembly 134 can include a fourth connector 136 configured to be coupled to the fluid source 109 and a valve 138 for selectively connecting the fluid source 109 to the injection lumen 114.

In the illustrated embodiment, the catheter 110 includes (i) a proximal region 115, (ii) an intermediate region 116 adjacent to and distal of the proximal region 115, (iii) a distal region 117 adjacent to and distal of the intermediate region 116, and (iv) a distal tip region 118 adjacent to and distal of the distal region 117 (collectively “the regions 115-118”). The lengths of the regions 115-118 can differ from one another. For example, in some embodiments the proximal region 115 has a first length, the intermediate region 116 has a second length less than the first length, the distal region 117 has a third length greater than the second length but less than the first length, and the distal tip region 118 has a fourth length less than the first, second, and third lengths. Additionally, some or all of the regions 115-118 can have varying hardness, durometer, flexibility, rigidity, thickness, and/or other properties. For example, in some embodiments the catheter 110 has a first hardness along the proximal region 115, a second hardness along the intermediate region 116 that is less than the first hardness, a third hardness along the distal region 117 that is less than the first hardness and the second hardness, and a fourth hardness in the distal tip region 118 that is greater than third hardness.

In some embodiments, the distal tip region 118 and/or another region of the catheter 110 includes a marker (e.g., marker band), such as a radiopaque marker configured to facilitate visualization of the position of the catheter 110 during a medical procedure (e.g., a clot removal procedure) using the catheter 110. In some embodiments, all or a portion of the distal region 117 and/or the distal tip region 118 can be configured to assume a curved shape. For example, the distal region 117 can comprise a shape memory material that is heat-set or otherwise configured to have a predetermined curved shape. More specifically, the distal region 117 can be configured to deflect such that the catheter 110 has a pre-shaped portion (e.g., along some or all of the distal region 117 and/or the distal tip region 118) as described in U.S. patent application Ser. No. 17/529,018, titled “CATHETERS HAVING SHAPED DISTAL PORTIONS, AND ASSOCIATED SYSTEMS AND METHODS,” and filed Nov. 17, 2021, which is incorporated by reference herein in its entirety.

In the illustrated embodiment, the clot treatment system 100 includes a dilator assembly 140 removably inserted through the valve 101 and the aspiration lumen 112 of the catheter 110. The dilator assembly 140 can include an elongated dilator 142 coupled to a first connector or first cap 144. The valve 101 can include a second connector or second cap 143 coupled to a proximal portion of the valve 101. The first cap 144 can be releasably coupled/locked to the second cap 143. Accordingly, inserting the dilator 142 into the catheter 110 can include moving the first cap 144 toward the second cap 143. In some embodiments, the first cap 144 can mate with the second cap 142 to lock the dilator assembly 140 to the valve 101 when the dilator 142 is fully seated/positioned within the catheter 110. In the fully seated position shown in FIG. 1, the dilator 142 is configured to extend past a distal tip or distal terminus 119 of the catheter 110. The dilator assembly 140 and the catheter 110 can be jointly advanced through the vasculature of a patient in this position with the dilator 142 providing an atraumatic distal or leading end during advancement. In some embodiments, the dilator assembly 140 can include features similar or identical to, and can function similarly or identically, to any of the dilator assemblies described in detail in U.S. patent application Ser. No. 18/156,944, titled “CLOT TREATMENT SYSTEMS WITH DILATOR LOCKING MECHANISMS, AND ASSOCIATED DEVICES AND METHODS,” and filed Jan. 19, 2023, which is incorporated by reference herein in its entirety.

The aspiration lumen 112 can extend from the first connector 102 to the distal terminus 119 of the catheter 110 and define a distal aspiration opening 145. Accordingly, the distal aspiration opening 145 can be coplanar with the distal terminus 119 of the catheter 110. The injection lumen 114 can extend from the second connector 104 toward the distal terminus 119 of the catheter 110 and define a distal injection opening 146. In the illustrated embodiment, the injection lumen 114 terminates proximal to the distal terminus 119 such that the injection opening 146 is positioned proximal to the aspiration opening 145. In other embodiments, the injection lumen 114 extends to the distal terminus 119 of the catheter 110 such that the aspiration opening 145 and the injection opening 146 are coplanar or substantially coplanar. In some embodiments, the aspiration lumen 112 has a larger dimension (e.g., diameter, radius, cross-sectional area) than a corresponding dimension of the injection lumen 114. For example, the aspiration lumen 112 can have a diameter of about 0.270 inch and the injection lumen 114 can have a diameter of about 0.039 inch. Accordingly, the aspiration lumen 112 can have a diameter of about 7 times greater, about 5 times greater, about 2 times greater, or more than 7 times greater than the injection lumen 114.

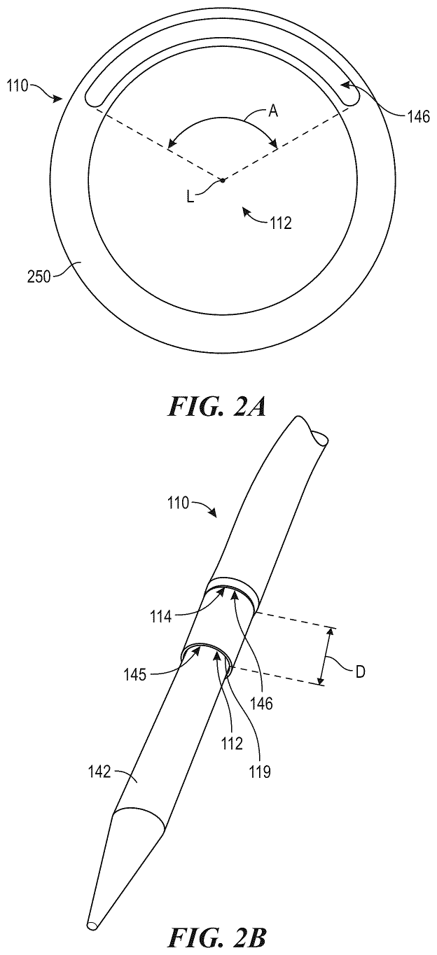

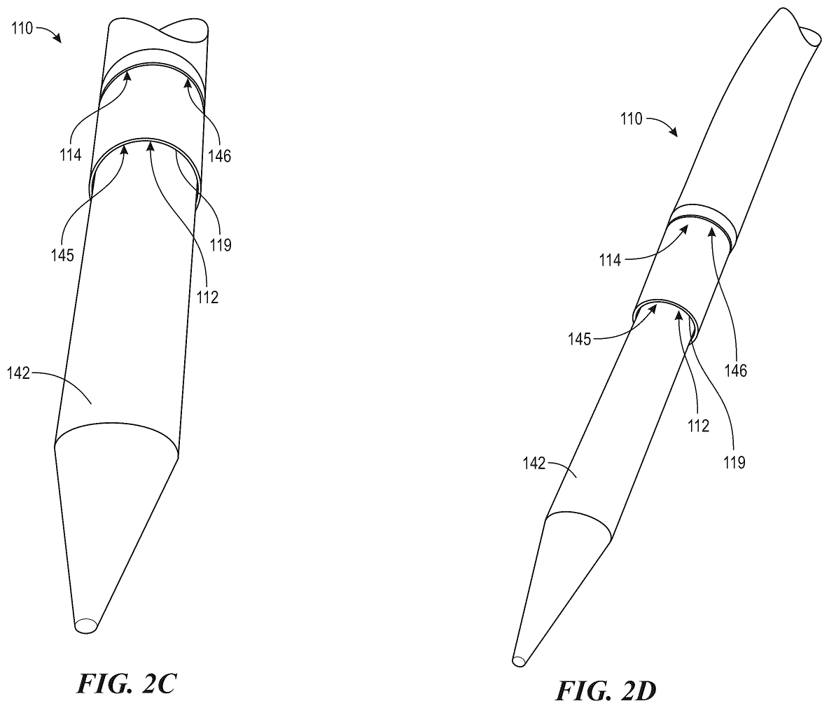

The aspiration lumen 112 can have a generally circular cross-sectional shape. The injection lumen 114 can have a generally circular cross-sectional shape, an elongate U-shaped cross-sectional shape, or other shape. In some embodiments, the injection lumen 114 comprises a single lumen while, in other embodiments, the injection lumen 114 comprises multiple (e.g., two, three, four, or more) parallel lumens. More particularly, for example, FIGS. 2A-5B illustrate different configurations (e.g., shapes, sizes, positioning) of the injection lumen 114 in accordance with embodiments of the present technology.

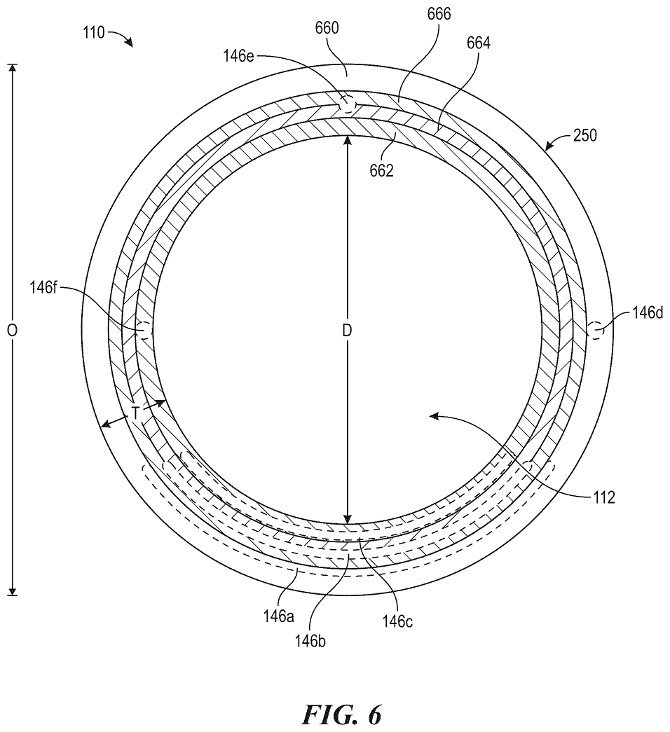

FIG. 2A is a cross-sectional view of the catheter 110 of FIG. 1 taken along the line 2A-2A in FIG. 1 in accordance with embodiments of the present technology. FIGS. 2B-2D are proximally-facing perspective views of a distal portion of the catheter 110 of FIGS. 1 and 2A with the dilator 142 of FIG. 1 inserted therein in accordance with embodiments of the present technology. Referring to FIG. 2A, the catheter 110 includes a wall 250 described in further detail below with reference to FIG. 6. The wall 250 encloses/defines the aspiration lumen 112. The injection lumen 114 can be formed in the wall 250 of the catheter 110 as described in greater detail below with reference to FIG. 6.

Referring to FIGS. 2A-2D, the aspiration lumen 112 has a generally circular cross-sectional shape, extends along a longitudinal axis L (FIG. 2A), and is configured to receive the dilator 142 therethrough. In the illustrated embodiment, the injection lumen 114 has an elongate U-shaped cross-sectional shape. More particularly, referring to FIG. 2A, the injection lumen 114 extends circumferentially through the wall 250 of the catheter 110 about the longitudinal axis L. In the illustrated embodiment, the injection lumen 114 extends about the longitudinal axis L by an angle A of about 120 degrees. In other embodiments, the injection lumen 114 can extend more or less circumferentially about the longitudinal axis L. For example, the angle A can be anywhere between about 110-140 degrees or between about 110-360 degrees.

Referring to FIGS. 2B-2D, the aspiration lumen 112 terminates at the aspiration opening 145 at the distal terminus 119 of the catheter 110, and the injection lumen 114 terminates at the injection opening 146 proximal of the distal terminus 119 of the catheter 110. Referring to FIG. 2B, the injection opening 146 can be spaced apart (e.g., spaced back) from the aspiration opening 145 by a distance D of between about 0.5-4.0 centimeters.

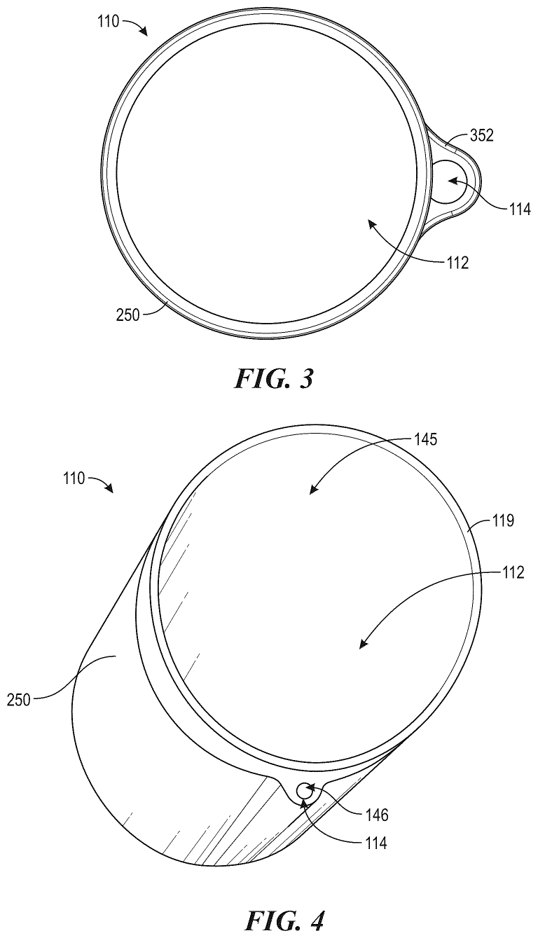

FIG. 3 is a cross-sectional view of the catheter 110 of FIG. 1 taken along the line 3-3 in FIG. 1 in accordance with additional embodiments of the present technology. In the illustrated embodiment, the catheter 110 includes the wall 250 and a tubular member 352 coupled to, attached to, and/or integrally formed with the wall 250. The wall encloses/defines the aspiration lumen 112, and the tubular member 352 at least partially encloses/defines the injection lumen 114. In some embodiments, the tubular member 352 is a separate circular tube or extrusion coupled to the wall 250 such that the injection lumen 114 has a generally tubular shape. In some embodiments, the tubular member 352 defines/encloses a first portion of the injection lumen 114 and the wall 250 encloses/defines a second portion of the injection lumen 114 such that the injection lumen 114 has a sector-like cross-sectional shape defined by the circular curvature of the tubular member 352 about the first portion and the circular curvature of the wall 250 about the second portion.

FIG. 4 is a proximally-facing perspective end-on view of the catheter 110 of FIGS. 1 and/or 3 in accordance with additional embodiments of the present technology. In the illustrated embodiment, the catheter 110 includes the wall 250 which encloses defines the aspiration lumen 112, and the injection lumen 114 is formed in the wall 250 of the catheter 110 as described in greater detail below with reference to FIG. 6. The aspiration lumen 112 and the injection lumen 114 can both have a circular cross-sectional shape. In the illustrated embodiment, the aspiration lumen 112 terminates at the aspiration opening 145 at the distal terminus 119 of the catheter 110, and the injection lumen 114 terminates at the injection opening 146 proximal of the distal terminus 119 of the catheter 110.

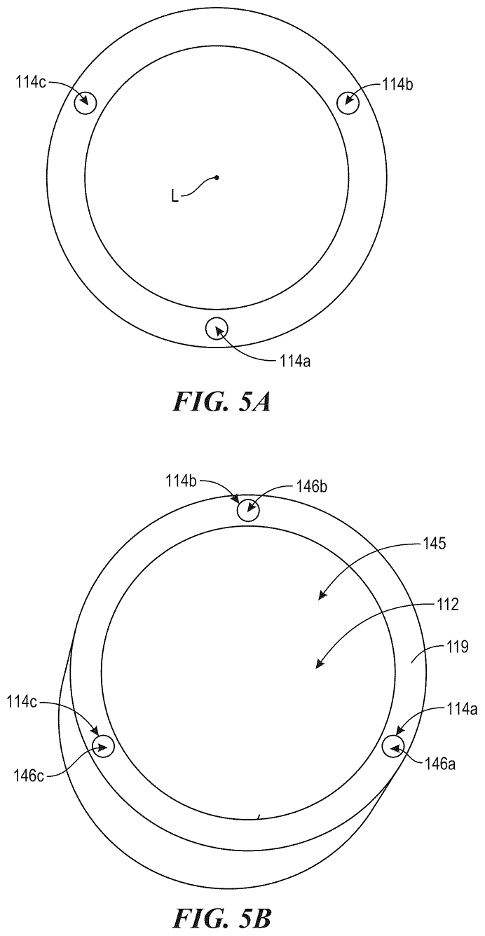

FIG. 5A is a cross-sectional view of the catheter 110 of FIG. 1 taken along the line 5A-5A in FIG. 1 in accordance with additional embodiments of the present technology. FIG. 5B is a proximally-facing perspective end-on view of the catheter 110 of FIGS. 1 and 5A in accordance with embodiments of the present technology. Referring to FIGS. 5A and 5B, the injection lumen 114 comprises multiple parallel injection lumens 114 (individually identified as first through third injection lumens 114a-c, respectively). Referring to FIGS. 1, 5A, and 5B, the injection lumens 114 can each be coupled to the second tubing assembly 130 via the second connector 104 to receive the fluid from the fluid source 109. Referring to FIGS. 5A and 5B, the catheter 110 includes the wall 250 which encloses defines the aspiration lumen 112, and the injection lumens 114 are formed in the wall 250 of the catheter 110 as described in greater detail below with reference to FIG. 6. While three of the injection lumens 114 are illustrated in FIGS. 5A and 5B and are equally spaced circumferentially about the longitudinal axis L (FIG. 5A) of the catheter 110 (e.g., by 120 degrees apart from one another), the catheter 110 can include more or fewer (e.g., two, four, five, more than five) of the injection lumens 114 and/or the injection lumens 114 can be spaced apart differently (e.g., irregularly). The aspiration lumen 112 and the injection lumens 114a-c can both have a circular cross-sectional shape. Referring to FIG. 5B, in the illustrated embodiment the aspiration lumen 112 terminates at the aspiration opening 145 at the distal terminus 119 of the catheter 110, and the first through third injection lumens 114a-c each terminate at a corresponding first through third injection opening 146a-c, respectively, at the distal terminus 119 of the catheter 110. In other embodiments, the injection openings 146 can be set back proximally from the distal terminus 119 of the catheter 110.

FIG. 6 is a cross-sectional view of the catheter 110 taken along the line 6-6 in FIG. 1 in accordance with embodiments of the present technology. In the illustrated embodiment, the wall 250 of the catheter 110 comprises an outer sheath 660 and an inner liner 662 extending through/defining each of the regions 115-118 (FIG. 1). The outer sheath 660 is positioned over (e.g., radially outside of) the inner liner 662. The outer sheath 660 can also be referred to as an outer jacket, an outer shaft, an outer layer, or the like, and the inner liner 662 can also be referred to as an inner layer, an inner sheath, an inner shaft, or the like. In the illustrated embodiment, the catheter 110 further includes an inner coil layer 664 (e.g., a first coil layer) and an outer coil layer 666 (e.g., a second coil layer) extending over/about the inner coil layer 664. The inner coil layer 664 and the outer coil layer 666 (collectively “the coil layers 664, 666”) can comprise a plurality of individual wires that are wound around the inner liner 662. In some embodiments, the wires extend around the inner liner 662 in a helical or spiral pattern about the longitudinal axis L (FIGS. 2A and 5A) of the catheter 110 in a first direction to form the inner coil layer 664, and the wires double back at or proximate to the distal terminus 119 (FIG. 1) to extend about the inner coil layer 664 in a helical or spiral pattern about the longitudinal axis L in a second direction to form the outer coil layer 666. The wires can thus self-terminate at and/or proximate to the distal terminus 119 of the catheter 110 where the wires transition from the inner coil layer 664 to the outer coil layer 666. The coil layers 664, 666 can extend along an entire length of the catheter 110 through each of the regions 115-118 (FIG. 1), or can extend only partially along the length of catheter 110 (e.g., in the distal region 117 and the distal tip region 118 of FIG. 1). The coil layers 664, 666 can be referred to collectively as a reinforcement structure or the like. In some embodiments, the catheter 110 can include features similar or identical to, and can function similarly or identically, to any of the catheters described in detail in U.S. patent application Ser. No. 18/463,960, titled “CATHETERS HAVING MULTIPLE COIL LAYERS, AND ASSOCIATED SYSTEMS AND METHODS,” and filed Sep. 8, 2023, which is incorporated by reference herein in its entirety.

The outer sheath 660 can be formed from a plastic material, elastomeric material, thermoplastic polyurethane (TPU), and/or thermoplastic elastomer (TPE) material. In some embodiments, the outer sheath 660 can be formed from a TPE manufactured by Arkema S.A., of Colombes, France, such as the TPEs manufactured under the trademark “Pebax.” In some embodiments, the outer sheath 660 can have a varying hardness (e.g., durometer), thickness, flexibility, rigidity, and/or other property in one or more of the different regions 115-118 as described in detail above with reference to FIG. 1. The outer sheath 660 can define an outer diameter O of the catheter 110. In some embodiments, the outer diameter O is greater than about 6 French, greater than about 10 French, greater than about 16 French, greater than about 20 French, greater than about 24 French, or greater. In some embodiments, the outer diameter O is about 8 French, about 16 French, about 20 French, about 24 French, or about 26 French. As used herein with reference to the outer diameter O, the term “about” means within plus or minus 1 French of the stated diameter.

The inner liner 662 defines/encloses the aspiration lumen 112 and, in some embodiments, can be formed of a lubricious material that facilitates the movement (e.g., distal advancement, proximal retraction) of various components through the aspiration lumen 112, such as the dilator assembly 140 (FIG. 1), delivery sheaths, pull members, guidewires, interventional devices, other aspiration catheters, and/or the like. In some embodiments, the inner liner 662 can be formed from a polymer material, a fluoropolymer material (e.g., polytetrafluoroethylene (PTFE)), and/or another material having a high degree of lubricity. The inner liner 662 defines an inner diameter D of the catheter 110. The thicknesses of the outer sheath 660, the inner liner 662, and the coil layers 664, 666 relative to one another and/or to the inner diameter D may not be to scale in FIG. 6. For example, one of ordinary skill in the art will understand that the thicknesses of these components are shown for clarity but that the wall 250 of the catheter 110 can be relatively thin compared to the inner diameter D. The inner diameter D can be less than the outer diameter O of the catheter 110 by a thickness T of the wall 250 of the catheter 110 comprising the combined thicknesses of the outer sheath 660, the inner liner 662, and the coil layers 664, 666. In some embodiments, the thickness T is about between about 0.2-2.0 mm, between about between about 1.0-2.0 mm, between about 1.2-1.4 mm, about 1.3 mm, between about 1.0-3.0 mm, and/or the like. Accordingly, in some embodiments, the inner diameter D is greater than about 6 French, greater than about 10 French, greater than about 16 French, greater than about 20 French, greater than about 24 French, or greater. In some embodiments, the inner diameter D is about 8 French, about 16 French, about 20 French, about 24 French, or about 26 French. In some embodiments, the outer diameter O is about 24 French, and the inner diameter D is about 20 French (e.g., the thickness T is about 1.3 mm). As used herein with reference to the inner diameter D, the term “about” means within plus or minus 1 French of the stated diameter. Referring to FIGS. 1 and 6, in some embodiments the inner diameter D of the inner liner 662 is the same in each of the regions 115-118 (FIG. 1) while, in other embodiments, the inner diameter D can vary along one or more of the regions 115-118.

In some embodiments, the inner liner 662 or the outer sheath 660 can be omitted. For example, the inner liner 662 can be omitted and the coil layers 664, 666 can be coupled to (e.g., fused to) the outer sheath 660.

As described above with reference to FIGS. 2A-5B, the injection lumen 114 can be formed in the wall 250 of the catheter 110. For example, the injection lumen 114 can comprise a tube, extrusion, elongate member, and/or the like that is formed/positioned between the coil layers 664, 666, between the inner coil layer 664 and the inner liner 662, and/or between the outer coil layer 666 and the outer sheath 660.

More specifically, FIG. 6 illustrates with dashed lines various configurations in which the injection lumen 114 can be formed in the wall 250 of the catheter 110, referenced as first through sixth injection lumens 114a-f, respectively. In the illustrated embodiment, the first injection lumen 114a extends between the outer sheath 660 and the outer coil layer 666 and has a generally U-like shape (e.g., as described in detail above with reference to FIGS. 2A-2D). The second injection lumen 114b extends between the coil layers 664, 666 and has a generally U-like shape. The third injection lumen 114c extends between the inner coil layer 664 and the inner liner 662 and has a generally U-like shape. The fourth injection lumen 114d extends between the outer sheath 660 and the outer coil layer 666 and has a generally circular shape (e.g., as described in detail above with reference to FIGS. 3-5B). The fifth injection lumen 114e extends between the coil layers 664, 666 and has a generally circular shape. And, the sixth injection lumen 114f extends between the inner coil layer 664 and the inner liner 662 and has a generally circular shape. The catheter 110 can include one or multiple of the injection lumens 114 having any suitable shape and position relative to the wall 250 of the catheter 110.

The injection lumen(s) 114 can be formed during manufacturing of the catheter 110 as, for example, described in detail in U.S. patent application Ser. No. 18/463,960, titled “CATHETERS HAVING MULTIPLE COIL LAYERS, AND ASSOCIATED SYSTEMS AND METHODS,” and filed Sep. 8, 2023, which is incorporated by reference herein in its entirety. For example, the injection lumen(s) 114 can be secured in position between the coil layers 664, 666 during manufacturing by (i) positioning the injection lumen(s) 114 over the inner coil layer 664 after winding the wires of the inner coil layer 664 about the inner liner 662 and then (ii) further winding the wires over the injection lumen(s) 114 and the inner coil layer 664 to form the outer coil layer 666. In some aspects of the present technology, the catheter 110 can be manufactured to include the injection lumen(s) 114 with a single machine setup due to the self-terminating ends of the coil layers 664, 666. In additional aspects of the present technology, the injection lumen(s) 114 can be formed within the catheter 110 without increasing or substantially increasing the thickness T of the wall 250 of the catheter 110 compared to conventional manufacturing methods.

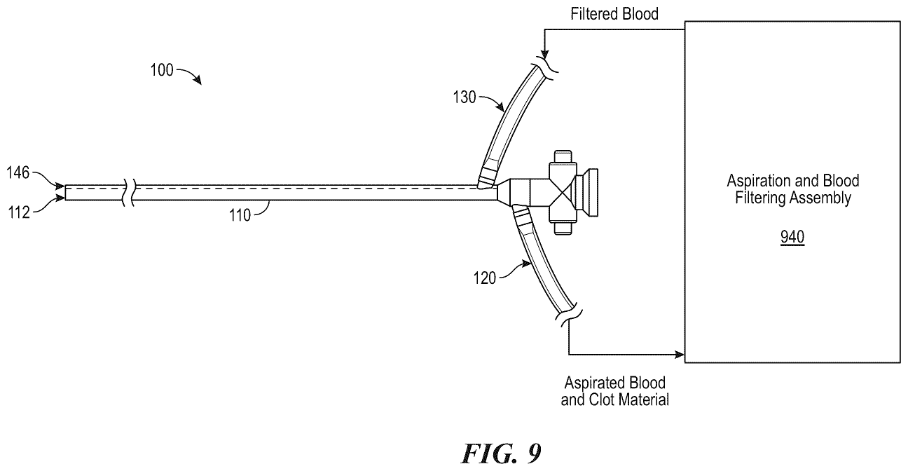

Referring to FIG. 1, in some embodiments the second tubing assembly 130 can be fluidly coupled to a source of filtered blood and the injection lumen 114 can be utilized to reinfuse the filtered blood into the vasculature of a patient. For example, FIG. 9 is a partially-schematic side view of the clot treatment system 100 configured as a clot aspiration and filtered blood reinfusion system in accordance with embodiments of the present technology. In the illustrated embodiment, the aspiration lumen 112 and the injection lumen 114 of the catheter 110 are fluidly coupled to an aspiration and blood filtering assembly 940 via the first tubing assembly 120 and the second tubing assembly 130, respectively. The aspiration and blood filtering assembly 940 is configured to aspirate blood and clot material through the aspiration lumen 112, filter the blood from the clot material, and reinfuse/return the filtered blood through the injection lumen 114 (which, accordingly, can be referred to as a reinfusion lumen).

In some embodiments, the aspiration and blood filtering assembly 940 comprises a first pressure source, a filter device, and a second pressure source. The first pressure source (e.g., a first syringe) can be fluidly coupled to the first tubing assembly 120 and activated to aspirate blood and clot material through the aspiration lumen 112 of the catheter 110, through the first tubing assembly 120, and into the first pressure source. The first pressure source can then be decoupled from the first tubing assembly 120, fluidly coupled to an inlet of the filter device, and activated to drive the blood and clot material into the filter device through the inlet. The filter device can include one or more filters configured to filter the blood from the clot material. The second pressure source (e.g., a second syringe) can be fluidly coupled to an outlet of the filter device and activated to draw the filtered blood from the filter device into the second pressure source. The second pressure source can then be decoupled from the outlet of the filter device, fluidly coupled to the second tubing assembly 130, and activated to drive the filtered blood through the second tubing assembly 130, through the injection lumen 114 of the catheter 110, and into the vasculature of the patient. Accordingly, in some embodiments the blood filtering assembly 940 can have some features generally similar or identical to and/or function generally similarly or identically to any of the aspiration and blood filtering devices described in detail in U.S. Pat. No. 11,559,382, filed Aug. 8, 2019, and titled “SYSTEM FOR TREATING EMBOLISM AND ASSOCIATED DEVICES AND METHODS,” which is incorporated herein by reference in its entirety.

In other embodiments, the aspiration and blood filtering assembly 940 can comprise one or more blood-filtering syringes or other devices configured to aspirate blood and clot material into the syringe via the aspiration lumen 112 and to filter the blood from the clot material for reinfusion through the reinfusion lumen 114. For example, the aspiration and blood filtering assembly 940 can have some features generally similar or identical to and/or function generally similarly or identically to any of the blood-filtering syringes/devices described in detail in U.S. patent application Ser. No. 18/192,855, filed Mar. 30, 2023, and titled “BLOOD-FILTERING DEVICES FOR USE WITH CLOT TREATMENT SYSTEMS,” which is incorporated herein by reference in its entirety.

In other embodiments, the aspiration and blood filtering assembly 940 can be an at least partially automated system configured to aspirate blood and clot material through the aspiration lumen 112, filter the clot material from the blood, and reinfuse/return the filtered blood through the injection lumen 114. For example, the aspiration and blood filtering assembly 940 can comprise one or more pumps (e.g., syringe, electric pumps, etc.), one or more filters, and one or more tubing sections configured to carry out the aforementioned functions. In some embodiments, the aspiration and blood filtering assembly 940 can have some features generally similar or identical to and/or function generally similarly or identically to any of the automated aspiration thrombectomy and blood reinfusion systems described in detail in U.S. Provisional Patent Application No. 63/675,087, filed Jul. 24, 2024, and titled “AUTOMATED CLOT ASPIRATION AND BLOOD REINFUSION SYSTEMS, AND ASSOCIATED DEVICES AND METHODS,” which is incorporated herein by reference in its entirety.

In some aspects of the present technology, because the aspiration lumen 112 and the injection lumen 114 are integrated into the same catheter 110, the system 100 can reduce the number of vascular access sites needed for a thrombectomy and blood reinfusion procedure as well as reducing the overall profile needed for the access site-improving procedure efficiency and reducing patient discomfort. Moreover, in some aspects of the present technology the aspiration and reinfusion operations of the aspiration and blood filtering assembly 940 can be independent such that the rate at which the aspiration and blood filtering assembly 940 aspirates material via the aspiration lumen 112 is independent from the rate at which the aspiration and blood filtering assembly 940 reinfuses filtered blood into the patient. That is, the rate at which fluid is removed from the patient need not be the same as the rate at which fluid is reintroduced into the patient such that the aspiration lumen 112 can be of different size (e.g., diameter) than the injection lumen 114.

### II. SELECTED EMBODIMENTS OF METHODS OF CLOT TREATMENT

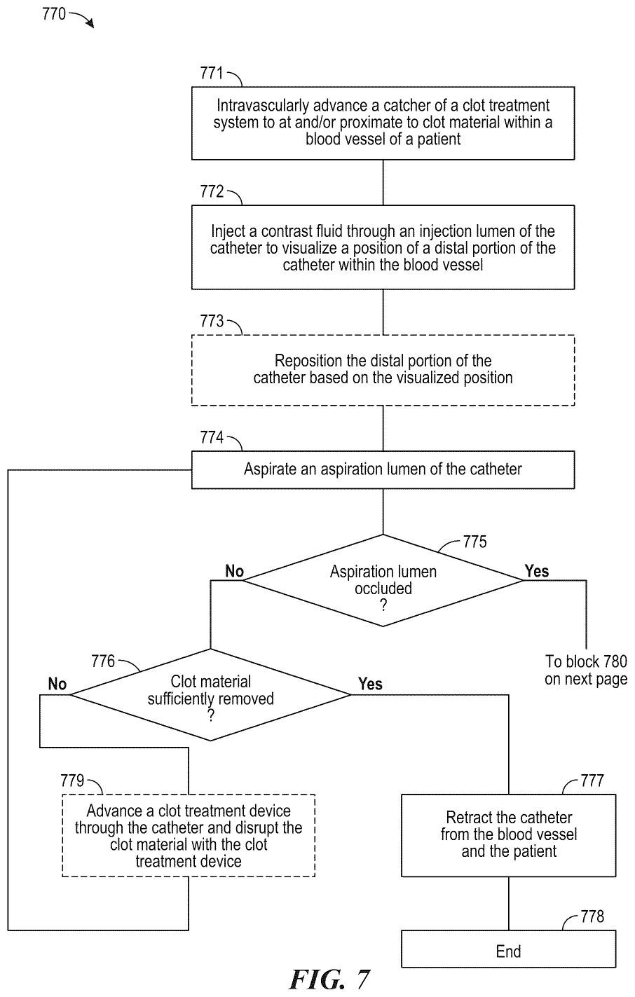

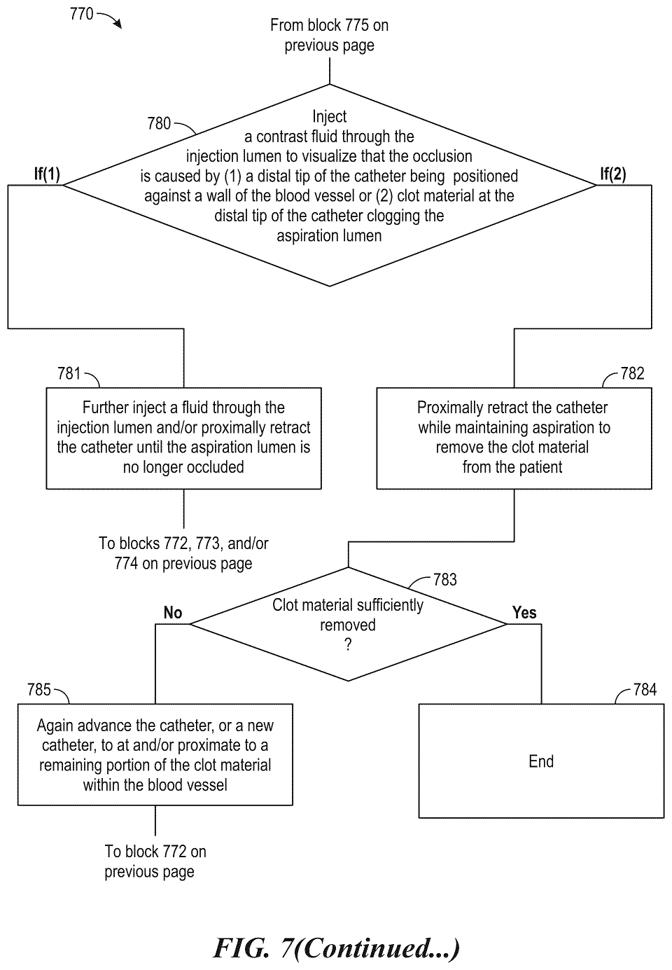

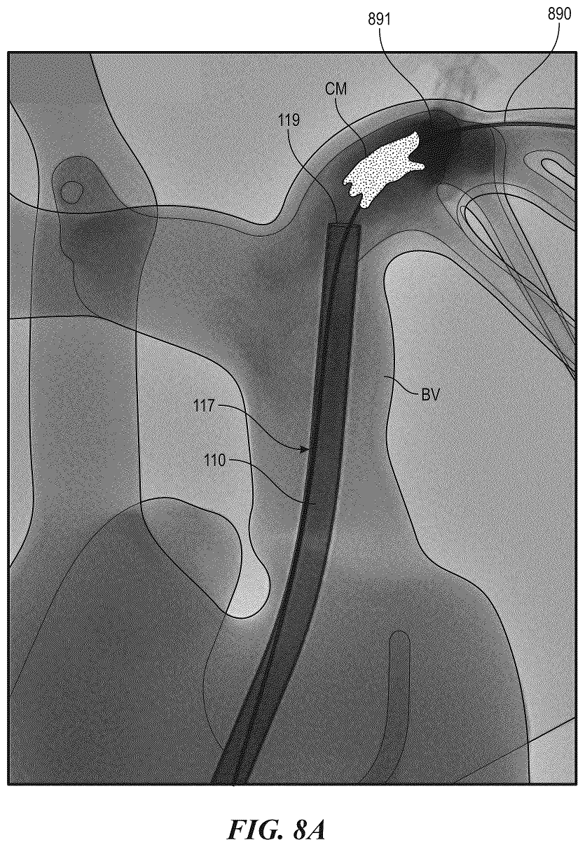

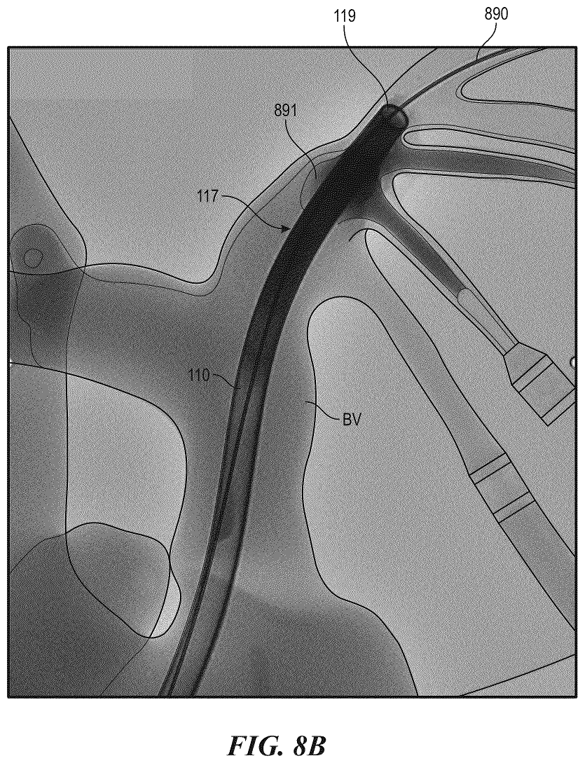

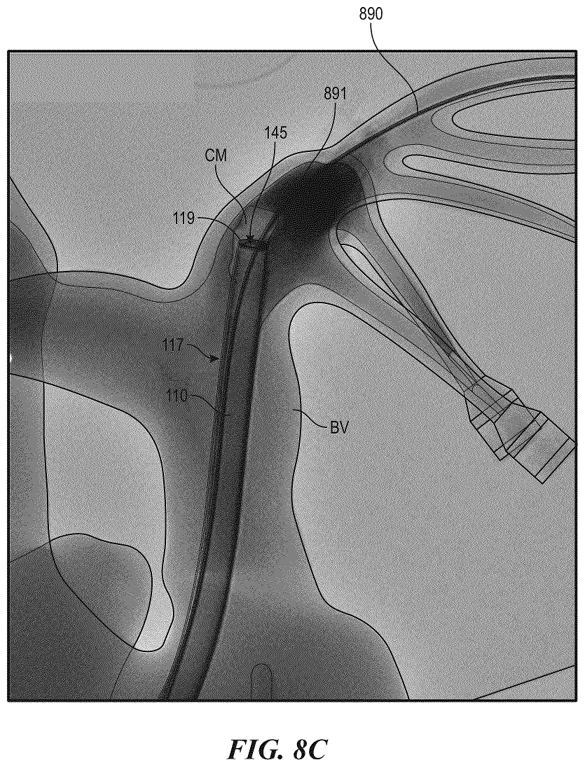

FIG. 7 is a flow diagram of a process or method 770 of treating clot material (e.g., removing clot material) within a patient, such as a human patient, in accordance with embodiments of the present technology. Although some features of the method 770 are described in the context of the clot treatment system 100 of FIG. 1 for the sake of illustration, one skilled in the art will readily understand that the method 770 can be carried out using other clot treatment systems. FIGS. 8A-8C are sides views (e.g., fluoroscopic images) of the distal region 117 of the catheter 110 of the clot treatment system 100 at various stages of the method 770 in accordance with embodiments of the present technology. In some embodiments, some aspects (e.g., blocks) of the method 770 can be generally similar or identical to any of the clot removal procedures disclosed in U.S. Pat. No. 11,559,382, filed Aug. 8, 2019, and titled “SYSTEM FOR TREATING EMBOLISM AND ASSOCIATED DEVICES AND METHODS,” which is incorporated herein by reference in its entirety.

Referring to FIG. 7, at block 771 the method 770 can include intravascularly advancing the catheter 110 of the clot treatment system 100 to at and/or proximate clot material within a blood vessel of a patient. In some embodiments, the catheter 110 is advanced through the blood vessel until the distal terminus 119 of the catheter 110 is positioned proximate to a proximal portion of the clot material. The position of the distal terminus 119 can be confirmed or located via visualization of a marker band or other marker positioned along the distal region 117 and/or along the distal tip region 118 using fluoroscopy or another imaging procedure (e.g., a radiographic procedure). In other embodiments, the distal terminus 119 can be positioned at least partially within the clot material or distal of the clot material. In some embodiments, the blood vessel can be a portion of left pulmonary artery, the temporal arteries, the inferior vena cava, or the right atrium. In some embodiments, the clot material can be a pulmonary embolism, deep vein thrombosis, clot in transit (CIT) within the right atrium, and/or the like.

Access to the pulmonary vessels can be achieved through the patient's vasculature, for example, via the femoral vein. In some embodiments, the clot treatment system 100 can include an introducer (e.g., a Y-connector with a hemostasis valve) that can be partially inserted into the femoral vein. A guidewire (e.g., a guidewire 890 shown in FIGS. 8A-8C) can be guided into the femoral vein through the introducer and navigated through the right atrium, the tricuspid valve, the right ventricle, the pulmonary valve, and into the main pulmonary artery. Depending on the location of the clot material, the guidewire can be guided to one or more of the branches of the right pulmonary artery and/or the left pulmonary artery. In some embodiments, the guidewire can be extended entirely or partially through the clot material. In other embodiments, the guidewire can be extended to a location just proximal of the clot material. After positioning the guidewire, the catheter 110 and the dilator assembly 140 can be placed over the guidewire and advanced to the position proximate to the clot material. The guidewire can extend through the aspiration lumen 112 or the injection lumen 114. The dilator assembly 140 can then be withdrawn from the aspiration lumen 112. In some embodiments, the guidewire can then be withdrawn while, in other embodiments, the guidewire can remain and can be used to guide other catheters (e.g., delivery catheters, additional aspiration guide catheters), interventional devices, etc., to the treatment site. It will be understood, however, that other access locations into the venous circulatory system of a patient are possible and consistent with the present technology. For example, the user can gain access through the jugular vein, the subclavian vein, the brachial vein, or any other vein that connects or eventually leads to the superior vena cava. Use of other vessels that are closer to the right atrium of the patient's heart can also be advantageous as it reduces the length of the instruments needed to reach the clot material.

At block 772, the method 770 can include injecting a contrast fluid (e.g., a contrast agent) through the injection lumen 114 of the catheter 110 to visualize a position and/or orientation of the distal region 117 and/or the distal tip region 118 of the catheter 110 within the blood vessel. FIG. 8A, for example, illustrates the distal region 117 of the catheter 110 positioned within a blood vessel BV with the distal terminus proximal 119 to clot material CM within the blood vessel BV. Referring to FIGS. 1 and 8A, the dilator assembly 140 has been removed from the aspiration lumen 112 (while the guidewire 890 extending through the aspiration lumen 112 or the injection lumen 114 remains) and a contrast fluid 891 has been injected into the blood vessel BV from the injection lumen 114. More specifically, the contrast fluid 891 can be driven from the fluid source 109, through the second tubing assembly 130, through the injection lumen 114, and out of the injection opening 146 into the blood vessel BV.

In some aspects of the present technology, an operator (e.g., physician) can inject only a bolus of the contrast fluid 891 having a relatively small volume through the injection lumen 114 for visualizing the position of the distal region 117 of the catheter 110. For example, the injection lumen 114 can be small in dimension relative to the aspiration lumen 112, and the injection opening 146 can be positioned at and/or proximate to the distal region 117 of the catheter 110 where visualization is desired. Accordingly, a smaller volume of the contrast fluid 891 can provide sufficient visualization, whereas a larger volume of the contrast fluid 891 would likely be needed to facilitate the same level of visualization if it were injected through, for example, the aspiration lumen 112.

The contrast fluid 891 can enable an operator (e.g., a physician) viewing the fluoroscopic or other image to visualize a position of the distal region 117 of the catheter 110 within the blood vessel BV. The operator can determine from the image whether the catheter 110 is optimally placed prior to aspiration (block 774). For example, the operator can determine that the catheter 110 is optimally located centrally within the blood vessel BV away from the walls of the blood vessel BV with the distal terminus 119 and the aspiration opening 145 proximate to the clot material CM. Optionally, based on the image, at block 773 the operator can reposition the distal region 117 of the catheter 110 within the blood vessel BV.

At block 774, the method 770 can include aspirating the aspiration lumen 112 of the catheter 110 to, for example, aspirate a portion of the clot material into and through the aspiration lumen 112. In some embodiments, the pressure source 106 is configured to generate (e.g., form, create, charge, build-up) a vacuum (e.g., negative relative pressure) and store the vacuum for subsequent application to the catheter 110. For example, after positioning the catheter 110 proximate the clot material, a user can first close the fluid control device 124 before generating the vacuum in the pressure source 106 by, for example, withdrawing the plunger of a syringe coupled to the third connector 126 of the first tubing assembly 120. In this manner, a vacuum is charged within the pressure source 106 (e.g., a negative pressure is maintained) before the pressure source 106 is fluidly connected to the aspiration lumen 112 of the catheter 110. To aspirate the aspiration lumen 112 of the catheter 110, the user can open the fluid control device 124 to fluidly connect the pressure source 106 to the aspiration lumen 112 and thereby apply or release the vacuum stored in the pressure source 106 to the aspiration lumen 112.

Opening of the fluid control device 124 instantaneously or nearly instantaneously applies the stored vacuum pressure to the first tubing assembly 120 and the aspiration lumen 112 of the catheter 110, thereby generating a suction pulse throughout the catheter 110. In particular, the suction is applied at the distal tip region 118 of the catheter 110 through the aspiration opening 145 with the intent to suck/aspirate at least a portion of the clot material into the aspiration lumen 112 of the catheter 110 from within the blood vessel. In some aspects of the present technology, pre-charging or storing the vacuum in the pressure source 106 before applying the vacuum to the aspiration lumen 112 of the catheter 110 is expected to generate greater suction forces and corresponding fluid flow velocities at and/or near the distal tip region 118 of the catheter 110 compared to simply activating the pressure source 106 while it is fluidly connected to the catheter 110. In other embodiments, the pressure source 106 can be activated while the fluid control device 124 is open to aspirate the clot material.

At decision block 775, the method 770 can include determining whether the aspiration lumen 112 of the catheter 110 is occluded (e.g., clogged) after aspiration (block 774). For example, if the aspiration lumen 112 does not become occluded during aspiration, blood and clot material will typically be aspirated proximally at least partially through the aspiration lumen 112, through the first tubing assembly 120, and into the pressure source 106. The pressure source 106 and/or the first tubing assembly 120 can be at least partially transparent to enable the operator to visually confirm, by the presence of the blood and clot material, that the aspiration did not occlude the aspiration lumen 112. For example, the pressure source 106 can be a clear syringe or clot container. In contrast, if the aspiration lumen 112 becomes occluded during aspiration, blood and clot material typically will not be aspirated through the aspiration lumen 112—or only a small amount of blood and clot material will be—and blood and clot material will therefore not be visible to the operator, confirming that the aspiration lumen 112 is occluded. In such instances, a cavitation can form in the aspiration lumen 112, the first tubing assembly 120, and/or the pressure source 106. In other embodiments, the clot treatment system 100 can include other devices or systems for detecting that the catheter 110 is occluded.

If the aspiration lumen 112 is not occluded at decision block 775, the method can proceed to decision block 776, where the method 770 can include determining whether the clot material has been sufficiently removed from the blood vessel. If the clot material is sufficiently removed at block 776, at block 777, the method 770 can include proximally retracting the catheter 110 from the blood vessel and the patient. In some embodiments, retracting the catheter 110 from the blood vessel and the patient can include retracting the catheter 110 into the lumen of a funnel catheter or other outer catheter. The method 770 can then end at block 778.

If the clot material is not sufficiently removed at block 776, the method 770 can include returning to block 774 to again aspirate the aspiration lumen 112 of the catheter 110 with the intent to suck/aspirate at least another portion of the clot material into the aspiration lumen 112 of the catheter 110 from within the blood vessel. In other embodiments, the method 770 can return to blocks 772 or 773 to optimize the positioning of the catheter 110 within the blood vessel before again aspirating the aspiration lumen 112 of the catheter 110. At block 779, the method 770 optionally includes, advancing a clot treatment device through the catheter 110 (e.g., through the aspiration lumen 112 and/or the injection lumen 114) for disrupting the remaining clot material before aspiration. In some embodiments, the clot treatment device can include features similar or identical to, and can function similarly or identically, to any of the clot treatment devices described in detail in U.S. Pat. No. 11,648,028, titled “METHODS AND APPARATUS FOR TREATING EMBOLISM,” and filed Jun. 26, 2020, and/or U.S. patent application Ser. No. 17/072,909, titled “SYSTEMS, DEVICES, AND METHODS FOR TREATING VASCULAR OCCLUSIONS,” and filed Oct. 16, 2020, each of which is incorporated by reference herein in its entirety.

If the aspiration lumen 112 is occluded at block 775, the method 770 can proceed to decision block 780. At decision block 780, the method 770 can include injecting a contrast fluid through the injection lumen 114 to visualize a cause of the occlusion and, more specifically for example, to determine whether the occlusion is caused by (1) the distal terminus 119 of the catheter 110 being positioned against (e.g., stuck against) a wall of the blood vessel or (2) clot material at the distal terminus 119 of the catheter 110 clogging the aspiration lumen 112 of the catheter 110. Regarding (1), rapid aspiration through the aspiration lumen 112 at block 774 can sometimes cause the blood vessel to collapse and/or for the aspiration through the aspiration opening 145 to be applied directly against the wall of the blood vessel rather than the clot material therein. Regarding (2), the clot material aspirated at block 774 can be large enough and/or of a consistency that it is not aspirated fully through the aspiration lumen 112 and instead is caught against the distal terminus 119 (e.g., partially within and partially outside the aspiration lumen 112) and occludes the aspiration opening 145. That is, the clot material can stick to the distal terminus 119 of the aspiration catheter as a “lollipop” or clump.

FIG. 8B illustrates scenario (1) of block 780 with the distal terminus 119 of the catheter 110 positioned against and occluded by a wall of the blood vessel BV after aspiration of the aspiration lumen 112 (block 774). Referring to FIGS. 1 and 8B, the contrast fluid 891 has been injected into the blood vessel BV from the injection lumen 114. The contrast fluid 891 enables the operator to visualize the distal region 117 of the catheter 110 and the surrounding anatomy in the fluoroscopic or other image. The operator of the clot treatment system 100 can determine from the image that the catheter 110 is occluded by the wall of the blood vessel BV rather than by the clot material. For example, the operator can determine that there is a lack of contrast fluid 891 distal to the distal terminus 119 of the catheter 110—indicating that there is no room or flow distal to the catheter 110 and therefore that the blood vessel BV is collapsed and/or that the catheter 110 is occluded by the wall of the blood vessel BV or clot is occluding that portion of the anatomy. In some embodiments described in detail above, the injection opening 146 of the injection lumen 114 is set back proximally from the distal terminus 119 and the aspiration opening 145 of the catheter 110. In some aspects of the present technology, this configuration can inhibit or even prevent the injection lumen 114 from becoming occluded by the wall of the blood vessel BV after aspiration. Accordingly, the contrast fluid 891 can be injected through and exit the injection lumen 114 even when the wall of the blood vessel BV occludes the aspiration lumen 112. In additional aspects of the present technology, an operator (e.g., physician) need only inject a bolus of the contrast fluid 891 having a relatively small volume through the injection lumen 114 for visualizing the cause of the occlusion, as described in detail above.

At block 781, the method 770 can then include further injecting a fluid through the injection lumen 114 and/or proximally retracting the catheter 110 until the aspiration lumen 112 is no longer occluded. For example, a fluid (e.g., a contrast fluid, saline, blood) can be injected through the injection lumen 114 to fill and expand the blood vessel to cause the wall of the blood vessel to release from the catheter 110. The fluid can be the same or different than the fluid injected through the injection lumen at blocks 772 and/or 780. In some aspects of the present technology, injecting the fluid through the separate injection lumen 114 rather than the aspiration lumen 112 reduces the risk of reintroducing any clot material captured within the aspiration lumen 112 into the blood vessel. Alternatively or additionally, the catheter 110 can be retracted proximally until the distal terminus 119 pulls away (e.g., becomes unstuck) from the wall of the blood vessel BV thereby clearing the occlusion. When the aspiration lumen 112 is no longer occluded, the unreleased vacuum pressure within the clot treatment system 100 will be applied to the no-longer-occluded aspiration lumen 112 thereby aspirating blood and/or clot material at least partially through the aspiration lumen 112, through the first tubing assembly 120, and into the pressure source 106. The operator can therefore visualize that the aspiration lumen 112 is no longer occluded by visually confirming the presence of blood and/or clot material in the proximal components of the clot treatment system 100 and/or by reading a pressure gauge coupled to the clot treatment system, as described in detail above with reference to block 775. When the aspiration lumen 112 is no longer occluded, the method 770 can return to any of blocks 772-774 to again position and then aspirate the aspiration lumen 112.

FIG. 8C illustrates scenario (2) of block 780 with the distal terminus 119 of the catheter 110 occluded by the clot material CM after aspiration of the aspiration lumen 112 (block 774). Referring to FIGS. 1 and 8B, the contrast fluid 891 has been injected into the blood vessel BV from the injection lumen 114. The contrast fluid 891 enables the operator to visualize the distal region 117 of the catheter 110 and the surrounding anatomy in the fluoroscopic or other image. The operator of the clot treatment system 100 can determine from the image that a portion of the clot material CM is captured on (e.g., stuck against) the distal terminus 119 of the catheter 110 and occludes the aspiration opening 145. In some embodiments described in detail above, the injection opening 146 of the injection lumen 114 is set back proximally from the distal terminus 119 and the aspiration opening 145 of the catheter 110. In some aspects of the present technology, this configuration can inhibit or even prevent the injection lumen 114 from becoming occluded by the clot material CM after aspiration. Accordingly, the contrast fluid 891 can be injected through and exit the injection lumen 114 even when the clot material CM occludes the aspiration lumen 112.

At block 782, the method 770 can then include proximally retracting the catheter 110 while maintaining aspiration to remove the clot material from the patient. The clot material is captured at and/or proximate to the aspiration opening 145 such that retraction of the catheter 110 also retracts the clot material. In some embodiments, retracting the catheter 110 and the captured clot material from the blood vessel and the patient includes retracting the catheter 110 and clot material into the lumen of a funnel catheter or other catheter.

At decision block 783, the method can include determining whether the clot material has been sufficiently removed from the blood vessel. If the clot material is sufficiently removed at decision block 783, at block 784 the method 770 can end. If the clot material is not sufficiently removed at decision block 783, the method 770 can proceed to block 785 to again advance the catheter 110, or a separate (e.g., new) catheter to at and/or proximate any of the clot material remaining within the blood vessel. In some embodiments, the catheter 110 can be cleaned (e.g., flushed) before being readvanced to the remaining clot material. The method can then return to block 772 to again position the catheter 110, aspirate the aspiration lumen 112, etc., to treat the remaining clot material.

In general, the method 770 permits the operator to directly visualize and determine the cause of an occlusion within the aspiration lumen 112 (e.g., whether the catheter 110 is engaged with and occluded by the wall of the blood vessel or occluded by clot material) via contrast fluid injected through the separate injection lumen 114. In contrast, many conventional clot removal techniques do not permit determination of the cause of an occlusion. For example, under fluoroscopy it can be difficult or impossible to tell what the cause of occlusion (e.g., cavitation) is because the area around the distal tip of the catheter (e.g., what the catheter is engaging) is not visible. In such systems, the catheter must be fully removed from the patient regardless of the cause of occlusion, cleaned, and subsequently reintroduced into the blood vessel. In some aspects of the present technology, the injection lumen 114 can be used to inject contrast agent to directly visualize the cause of occlusion as described in detail above with reference to block 781. If the catheter 110 is merely engaged with the wall of the blood vessel, the operator can inject fluid through the injection lumen 114 and/or slightly retract the catheter 110 to release the wall of the blood vessel from the catheter 110 and clear the occlusion. Notably, the operator need not retract the catheter 110 fully from the patient to clear the occlusion-reducing procedure time. Additionally, the injection lumen 114 is separate from the aspiration lumen 112 such that fluid injection through the injection lumen 114 minimizes the risk of reintroducing clot material into the blood vessel of the patient.

In some embodiments, the various blocks 771-785 of the method 770 can be performed in a different order, and/or some of the blocks 771-785 can be omitted. For example, in some embodiments blocks 772 and 773 can be omitted.

### III. ADDITIONAL EXAMPLES

Several aspects of the present technology are set forth in the following examples:

1. A clot treatment system, comprising:
a catheter having a distal tip configured to be intravascularly positioned proximate to clot material within a blood vessel, wherein the catheter includes an aspiration lumen having a distal aspiration opening and an injection lumen having a distal injection opening;
a pressure source fluidly coupled to the aspiration lumen, wherein the pressure source is configured to generate negative pressure within the aspiration lumen to aspirate at least a portion of the clot material through the distal aspiration opening; and
a fluid source fluidly coupled to injection lumen, wherein the fluid source is configured to inject a fluid through the injection lumen and out of the distal injection opening.

2. The clot treatment system of example 1 wherein the fluid is a contrast agent visible under fluoroscopic imaging.

3. The clot treatment system of example 1 wherein the fluid is filtered blood.

4. The clot treatment system of any one of examples 1-3 wherein the catheter extends along a longitudinal axis, wherein the catheter defines a wall, wherein the wall encloses the aspiration lumen, and wherein the injection lumen extends through the wall along the longitudinal axis.

5. The clot treatment system of example 4 wherein the injection lumen has a circular cross-sectional shape.

6. The clot treatment system of example 4 wherein the injection lumen has a generally U-like shape extending about the longitudinal axis.

7. The clot treatment system of example 6 wherein the injection lumen extends between about 110-140 degrees about the longitudinal axis.

8. The clot treatment system of any one of examples 1-7 wherein the injection lumen comprises multiple parallel lumens.

9. The clot treatment system of any one of examples 1-8 wherein the distal injection opening is positioned proximal to the distal aspiration opening.

10. The clot treatment system of example 9 wherein the distal aspiration opening is coplanar with the distal tip.

11. The clot treatment system of example 10 wherein the aspiration lumen has a first cross-sectional area, and wherein the injection lumen has a second cross-sectional area less than the first cross-sectional area.

12. A method of treating clot material within a blood vessel of a patient, the method comprising:
intravascularly positioning a distal tip of a catheter proximate to the clot material within the blood vessel;
generating, via an aspiration source, negative pressure within an aspiration lumen of the catheter;
determining that the aspiration lumen is occluded;
injecting a first fluid through an injection lumen of the catheter separate from the aspiration lumen to visualize that the aspiration lumen is occluded by either a wall of the blood vessel or the clot material;
if the aspiration lumen is occluded by the wall of the blood vessel, injecting a second fluid through the injection lumen into the blood vessel and/or proximally retracting the catheter until the aspiration lumen is no longer occluded; and
if the aspiration lumen is occluded by the clot material, proximally retracting the catheter and the clot material from within the blood vessel and out of the patient.

13. The method of example 12 wherein the first fluid is the same as the second fluid.

14. The method of example 12 or example 13 wherein the first fluid and the second fluid comprise a contrast fluid visible under fluoroscopic imaging.

15. The method of any one of examples 12-14 wherein the aspiration lumen terminates distally at the distal tip of the catheter, and wherein the injection lumen terminates distally proximal of the distal tip of the catheter.

16. The method of any one of examples 12-15 wherein, if the aspiration lumen is occluded by the wall of the blood vessel, the method further comprises again generating, via the aspiration source, negative pressure within the aspiration lumen of the catheter after the aspiration lumen is no longer occluded.

17. A method of treating clot material within a blood vessel of a patient, the method comprising:
intravascularly positioning a distal tip of a catheter proximate to the clot material within the blood vessel;
generating, via an aspiration source, negative pressure within an aspiration lumen of the catheter;
determining that the aspiration lumen is occluded;
injecting a first fluid through an injection lumen of the catheter separate from the aspiration lumen to visualize that the aspiration lumen is occluded by a wall of the blood vessel; and
injecting a second fluid through the injection lumen into the blood vessel and/or proximally retracting the catheter until the aspiration lumen is no longer occluded by the wall of the blood vessel.

18. The method of example 17 wherein the first fluid and the second fluid comprise a contrast fluid visible under fluoroscopic imaging.

19. The method of example 17 or example 18 wherein the method comprises injecting the second fluid through the injection lumen into the blood vessel until the aspiration lumen is no longer occluded by the wall of the blood vessel.

20. The method of any one of examples 17-19 wherein the method comprises proximally retracting the catheter until the aspiration lumen is no longer occluded by the wall of the blood vessel.

21. The method of any one of examples 17-20 wherein the method further comprises again generating, via the aspiration source, negative pressure within the aspiration lumen of the catheter after the aspiration lumen is no longer occluded by the wall of the blood vessel.

22. The method of any one of examples 17-21 wherein the aspiration lumen terminates distally at the distal tip of the catheter, and wherein the injection lumen terminates distally proximal of the distal tip of the catheter.

23. A method of treating clot material within a blood vessel of a patient, the method comprising:
intravascularly positioning a distal tip of a catheter proximate to the clot material within the blood vessel;
generating, via an aspiration source, negative pressure within an aspiration lumen of the catheter;
determining that the aspiration lumen is occluded;
injecting a first fluid through an injection lumen of the catheter separate from the aspiration lumen to visualize that the aspiration lumen is occluded by the clot material; and
proximally retracting the catheter and the clot material from within the blood vessel and out of the patient.

24. The method of example 23 wherein proximally retracting the catheter and the clot material comprises proximally retracting the catheter and the clot material into the lumen of a funnel catheter and out of the patient.

25. The method of example 23 or example 24 wherein the aspiration lumen terminates distally at the distal tip of the catheter, and wherein the injection lumen terminates distally proximal of the distal tip of the catheter.

### IV. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. (canceled)

2. The method of claim 12 wherein the first fluid is a contrast agent visible under fluoroscopic imaging.

3. The method of claim 12 wherein the second fluid is filtered blood.

4. The method of claim 12 wherein the catheter extends along a longitudinal axis, wherein the catheter defines a wall, wherein the wall encloses the aspiration lumen, and wherein the injection lumen extends through the wall along the longitudinal axis.

5. The method of claim 4 wherein the injection lumen has a circular cross-sectional shape.

6. The method of claim 4 wherein the injection lumen has a generally U-like shape extending about the longitudinal axis.

7. The method of claim 6 wherein the injection lumen extends between about 110-140 degrees about the longitudinal axis.

8. The method of claim 12 wherein the injection lumen comprises multiple parallel lumens.

9. (canceled)

10. (canceled)

11. The method of claim 12 wherein the aspiration lumen has a first cross-sectional area, and wherein the injection lumen has a second cross-sectional area less than the first cross-sectional area.

12. A method of treating clot material within a blood vessel of a patient, the method comprising:
intravascularly positioning a distal tip of a catheter proximate to the clot material within the blood vessel;
generating, via an aspiration source, negative pressure within an aspiration lumen of the catheter;
determining that the aspiration lumen is occluded;
injecting a first fluid through an injection lumen of the catheter separate from the aspiration lumen to visualize that the aspiration lumen is occluded by either a wall of the blood vessel or the clot material;
if the aspiration lumen is occluded by the wall of the blood vessel, injecting a second fluid through the injection lumen into the blood vessel and/or proximally retracting the catheter until the aspiration lumen is no longer occluded; and
if the aspiration lumen is occluded by the clot material, proximally retracting the catheter and the clot material from within the blood vessel and out of the patient.

13. The method of claim 12 wherein the first fluid is the same as the second fluid.

14. The method of claim 13 wherein the first fluid and the second fluid comprise a contrast fluid visible under fluoroscopic imaging.

15. The method of claim 12 wherein the aspiration lumen terminates distally at the distal tip of the catheter, and wherein the injection lumen terminates distally proximal of the distal tip of the catheter.

16. A method of treating clot material within a blood vessel of a patient, the method comprising:
intravascularly positioning a distal tip of a catheter proximate to the clot material within the blood vessel;
generating, via an aspiration source, negative pressure within an aspiration lumen of the catheter;
determining that the aspiration lumen is occluded;
injecting a first fluid through an injection lumen of the catheter separate from the aspiration lumen to visualize that the aspiration lumen is occluded by a wall of the blood vessel; and
injecting a second fluid through the injection lumen into the blood vessel and/or proximally retracting the catheter until the aspiration lumen is no longer occluded by the wall of the blood vessel.

17. The method of claim 16 wherein the first fluid and the second fluid comprise a contrast fluid visible under fluoroscopic imaging.

18. The method of claim 16 wherein the method comprises injecting the second fluid through the injection lumen into the blood vessel until the aspiration lumen is no longer occluded by the wall of the blood vessel.

19. The method of claim 16 wherein the method comprises proximally retracting the catheter until the aspiration lumen is no longer occluded by the wall of the blood vessel.

20. The method of claim 16 wherein the method further comprises again generating, via the aspiration source, negative pressure within the aspiration lumen of the catheter after the aspiration lumen is no longer occluded by the wall of the blood vessel.

21. The method of claim 16 wherein the aspiration lumen terminates distally at the distal tip of the catheter, and wherein the injection lumen terminates distally proximal of the distal tip of the catheter.

22. (canceled)

23. The method of claim 12 wherein proximally retracting the catheter and the clot material comprises proximally retracting the catheter and the clot material into the lumen of a funnel catheter and out of the patient.

24. (canceled)
